(19) United States
(12) Patent Application Publication
(10) Pub. No.: US 2021/0299420 A1
(43) Pub. Date: Sep. 30, 2021

(54) A DEVICE WITH SHIELD FOR ADMINISTERING THERAPEUTIC SUBSTANCES USING A HIGH VELOCITY LIQUID-GAS STREAM

(71) Applicant: TAV - TECH LTD, Yehud (IL)

(72) Inventors: Ronen SOBEL, Kochav Ya'ir (IL); Alexander POLYAKOV, Qidmat Tsvi (IL)

(73) Assignee: TAV - TECH LTD, Yehud (IL)

(21) Appl. No.: 17/260,232

(22) PCT Filed: Aug. 25, 2019
(86) PCT No.: PCT/IL2019/050950

§ 371 Date: Jan. 14, 2021

Related U.S. Application Data
(60) Provisional application No. 62/726,262, filed on Sep. 2, 2018.

(51) Int. Cl. A61M 35/00

(52) U.S. Cl. CPC A61M 35/003; A61M 11/06

(57) ABSTRACT

A device and system for treatment of tissue by direct application thereto of therapeutic substances in the form of a stream of therapeutic droplets carried in a high velocity gas. The high velocity gas is produced by accelerating a flow of gas through at least one gas discharge nozzle. At least one flow of therapeutic substance is introduced into the high velocity gas through at least one liquid discharge nozzle, thereby fragmenting the at least one flow of therapeutic liquid into a stream of therapeutic droplets. The stream is accelerated to a velocity similar to the velocity of the gas discharge flow. The accelerated therapeutic droplet stream is then applied to a tissue for therapeutic treatment. The device and system contain a gas stream shield generator that provides a shield which prevents rebounding and dispersing droplets produced after impinging on the tissue from travelling in the direction of a user.

### FIELD OF THE INVENTION

The present invention relates, generally, to devices for administering therapeutic substances to biological tissue, and, more specifically, to devices for applying a high velocity therapeutic liquid-gas stream for administering such substances to body tissue often in predefined dosages and concentrations.

### BACKGROUND OF THE INVENTION

Devices for dermal abrasion of exposed in vivo tissue are known in the art. One such device is described in U.S. Pat. No. 7,901,373 and another in U.S. Pat. No. 9,233,207, included herein by reference in their entirety. These documents also provides a general overview of the prior art of dermal abrasion and dermal abrasion devices.

Disclosed in the above referenced documents are devices for dermal abrasion employing a high-velocity liquid-gas streaming mist. The disclosed devices are particularly successful in overcoming the difficulty of stagnant boundary layers. When a fluid stream is employed to irrigate a tissue surface, a boundary layer is formed which is characterized by having a fluid velocity which decreases sharply adjacent to the flow surface, being virtually zero at the tissue surface. As a result, particles which are smaller than the thickness of the boundary layer of the fluid stream are often difficult or impossible to remove. The smallest particles in the boundary layer exhibit a drag resistance of a magnitude sufficient for these particles to remain attached to the surface and to resist being swept away by the fluid stream. The devices disclosed in the above referenced documents overcome this difficulty, its liquid-gas streaming mist producing a boundary layer of minimal to negligible thickness.

The above mentioned devices and other prior art devices require relatively large liquid and gas sources, suitable for use with a plurality of patients. These sources are positioned distant from the device necessitating the use of connecting tubes which inter alia impede use, especially one-hand use, of the devices.

### Definitions

In the discussion herein below:

The term “distal” refers to the position on the devices discussed herein furthest from the user that is the portion closest to the nozzle arrangement of the devices. The term “proximal” refers to the position on the devices closest to the user that is the portion furthest from the nozzle arrangement of the devices.

The terms “cleanse”, “cleaning” and variants thereof in the discussion herein below, refers to the removal of solid contaminants, such as fibers, dust, sand particles, and the like, as well as the removal of organic matter, such as pus, fats, and the like from the surface of tissue being cleaned and/or being treated with therapeutic substances. The term “cleanse” includes lavage of hollow organs of the body.

The term “tissue” as used herein can refer to either human or animal tissue.

The term “slit” of the gas stream shield generator may at times be called “openings” “holes” and the like. It should readily be apparent to the reader when a slit is being discussed or when the slit's exit hole or exit opening is being discussed.

The term “air” when used herein and in the claims can also refer to other relevant benign gases such as nitrogen which can be used for the same purpose. Similarly, the term “gasl” includes air, a mixture of gases, and other relatively benign gases such as nitrogen which can be used for the same purpose. This is true of the gases flowing through both the gas stream shielding generator and the nozzle arrangement of the devices discussed herein.

The term “therapeutic substance” when used herein includes liquids, and solids dispersed in at least one liquid carrier.

### SUMMARY OF THE INVENTION

An object of the present invention is to provide a device for treating biological tissue with therapeutic substances wherein the microdroplets generated by the device will not disperse or rebound in the direction of the user.

Another object is providing for a device that can reduce the amount of therapeutical materials required.

In one aspect of the present invention there is provided a device for administering a therapeutic substance to tissue for use with a pressurized gas source. The device includes:a housing having a liquid therapeutic substance inlet port; a gas inlet port connected to the pressurized gas source; a stream jet delivery nozzle arrangement in fluid flow communication with the gas inlet port and in fluid flow communication with the therapeutic substance inlet port, the therapeutic substance being discharged from the stream jet delivery nozzle arrangement into an elevated velocity flow of gas discharged from the delivery nozzle arrangement where upon the substance forms microdroplets which when impinging upon the tissue to be treated rebounds and disperses therefrom; and a gas stream shield generator comprised of a plurality of slits, wherein pressurized gas passes through the slits providing gas streams external to the nozzle arrangement, the gas streams forming an envelope that reduces the dispersal of the rebounded droplets resulting from impinging on the tissue, thereby shielding the user.

In some embodiments of the device, the gas stream shield generator includes an insert and a wall section of the housing or a wall section of the proximal portion of the nozzle arrangement. The insert is disposed within the wall section and constructed so that there are a plurality of identical spacers circumscribing an exterior side of the insert, spacing the insert from the wall section, thereby generating the plurality of slits through which the pressurized gas passes.

In some embodiments of the device, the number of slits are from 2 to 16 slits. The plurality of slits are symmetrically disposed on the distal edge of the shield generator insert, and each slit is equidistant from its nearest neighbor slits.

In still other embodiments of the device, the area of each slit is between 0.075 millimeters squared (“mm²”) and 0.5 mm². In some embodiments of the device, the area of each slit is between 0.1 mm² and 0.2 mm².

In some embodiments of the device the slits are shaped as circular arc sections.

In yet other embodiments of the device, the device further includes one or more therapeutic substance supply assemblies mounted onto the housing. Each therapeutic substance supply assembly is configured for receiving one or more containers containing a predefined quantity or concentration of liquid therapeutic substance.

In still other embodiments of the device, the liquid therapeutic substance inlet port is in fluid flow communication with the therapeutic substance supply assembly and also in fluid flow communication with the stream jet delivery nozzle arrangement.

In another embodiment of the device, the stream jet delivery nozzle arrangement includes: one or more gas discharge nozzles arranged to receive a flow of pressurized gas from the gas inlet port and configured to accelerate the flow of gas so as to discharge it at an elevated velocity; and one or more liquid discharge nozzles arranged to receive a flow of liquid therapeutic substance from a therapeutic substance supply assembly and operative to discharge the flow of therapeutic substance into the elevated velocity flow of gas, thereby to accelerate the velocity of the discharged liquid therapeutic substance as a stream of accelerated therapeutic droplets and to discharge the stream of accelerated therapeutic droplets towards a tissue mass for treatment by the therapeutic substance.

In another aspect of the present invention there is provided a system for administering a therapeutic substance to tissue. The system includes: a pressurized gas source; one or more containers containing a predefined quantity or concentration of a liquid therapeutic substance;

and a device. The device includes: a housing having a liquid therapeutic substance inlet port; a gas inlet port connected to the pressurized gas source; a stream jet delivery nozzle arrangement in fluid flow communication with the gas inlet port and in fluid flow communication with the liquid therapeutic substance, the liquid therapeutic substance being discharged from the stream jet delivery nozzle arrangement into an elevated velocity flow of gas discharged from the delivery nozzle arrangement which upon impinging the tissue to be treated rebounds and disperses therefrom; and a gas stream shield generator comprised of a plurality of slits, wherein pressurized gas passes through the slits providing gas streams external to the nozzle arrangement, the gas streams forming an envelope that reduces dispersal of the droplets resulting from impinging on the tissue, thereby shielding the user.

In yet another aspect of the present invention there is provided a gas stream shield generator. The generator includes a wall of a nozzle arrangement and/or hand piece with an insert disposed therein for producing a plurality of slits therebetween. The generator has a generally truncated conical shape with a wider end proximal to a gas source and a narrower end distal from the gas source. The slits have openings to the ambient at the distal end of the generator where pressurized gas passes through the slits providing gas streams external to the nozzle arrangement. The gas streams form an envelope that reduces dispersal of microdroplets generated by the nozzle arrangement thereby shielding a user from the liquid.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1 is a perspective view of a prior art device for administering therapeutic substances to tissue;

FIG. 2 is a schematic side view of the prior art device of FIG. 1;

FIGS. 3 and 4 are enlarged schematic and graphical representations, respectively, of a delivery nozzle arrangement of the prior art device seen in FIGS. 1 and 2;

FIG. 5 is a schematic view of a flow of stream droplets discharging from the prior art delivery nozzle arrangement as seen in FIG. 4 against a surface to which therapeutic substances are to be administered;

FIG. 6 is a schematic view of a prior art nozzle arrangement having multiple gas and liquid discharge nozzles;

FIGS. 7A-7C are perspective, side, and top views, respectively, of a device for administering therapeutic substances to tissue, the device constructed and operative in accordance with an embodiment of the present invention;

FIGS. 7D-7E are perspective and side views, respectively, of another device for administering therapeutic substances to tissue, constructed and operative substantially in accordance with the embodiment of the present invention shown in FIGS. 7A-7C;

FIGS. 8A and 8B show cut-away side views of a device of the present invention with and without a gas stream shield, respectively;

FIG. 8C is an isometric view of the gas stream shield generator;

FIG. 8D is a head-on view of the device in FIG. 8B which uses a gas stream shield showing the slit openings through which the gas streams forming the gas stream shield are emitted;

FIG. 8E is an isometric view of the device in FIGS. 8A-8D showing the gas streams forming an essentially cylindrical shield around the dispersing microdroplets, the shield essentially concentric with the liquid discharge nozzle of the device; and

FIG. 8F shows another schematic view of the gas stream shield used with the device of the invention.

### DETAILED DESCRIPTION OF THE INVENTION

The present invention relates to a device for administering therapeutic substances to tissue by directing a liquid-gas stream of droplets containing one or more therapeutic substances. The device includes two elements, a housing and a stream jet nozzle arrangement, the latter mechanically connected to the housing or integrally formed therefrom. The present invention is constructed to prevent microdroplets rebounding and/or scattering in the direction of the user after impinging on the tissue of a patient being treated.

The liquid-gas stream consists of one or more therapeutic liquids provided at a high velocity, generally within the mid sub-sonic range. While the average droplet velocity is in the mid sub-sonic range, some droplets may be accelerated to supersonic speeds.

To achieve these high velocities, gas is discharged from a device containing a stream jet nozzle arrangement, the arrangement containing one or more converging-diverging gas nozzles configured to accelerate the flow of gas so as to discharge it at an elevated velocity. A low rate of flow of therapeutic liquid is discharged into the elevated velocity flow of gas, thereby accelerating the discharged therapeutic liquid as a therapeutic stream of accelerated droplets. The volumetric rate of flow of therapeutic liquid from the device is relatively low, thereby essentially preventing the formation of a virtually stagnant liquid boundary layer on the surface of the tissue to which the therapeutic substances is being administered.

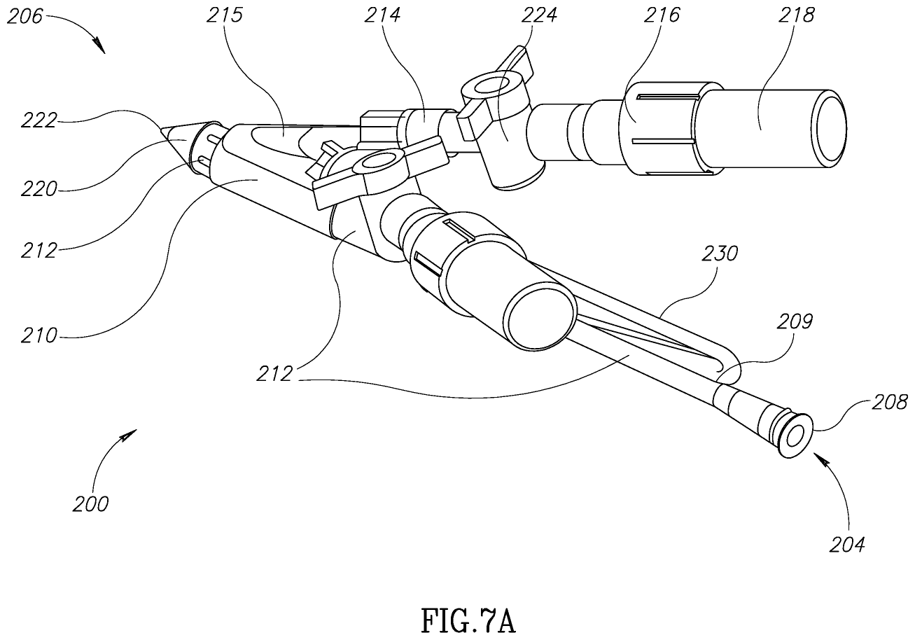

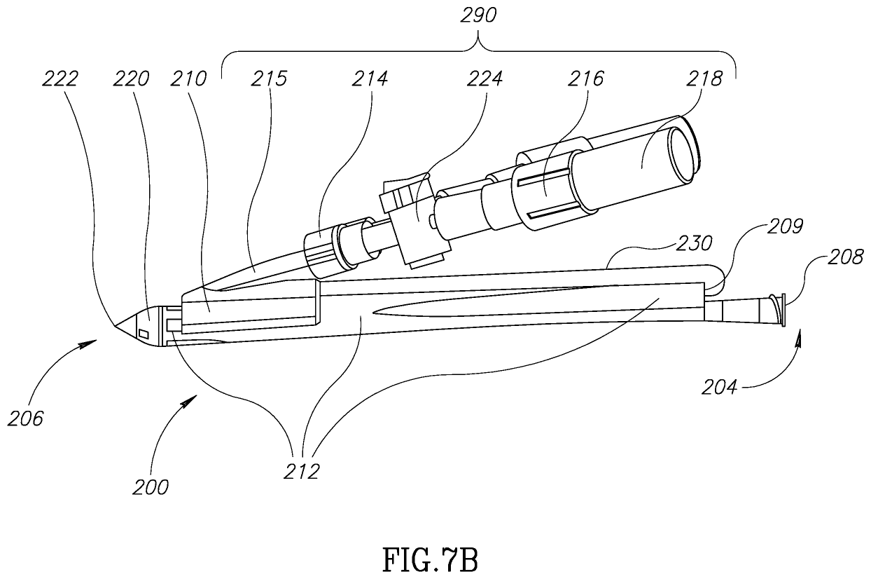

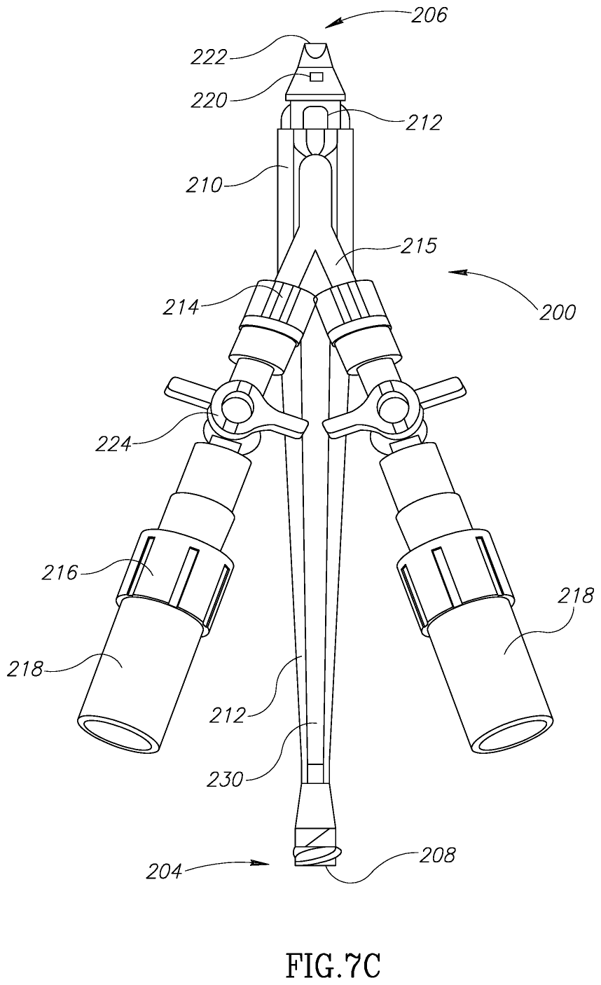

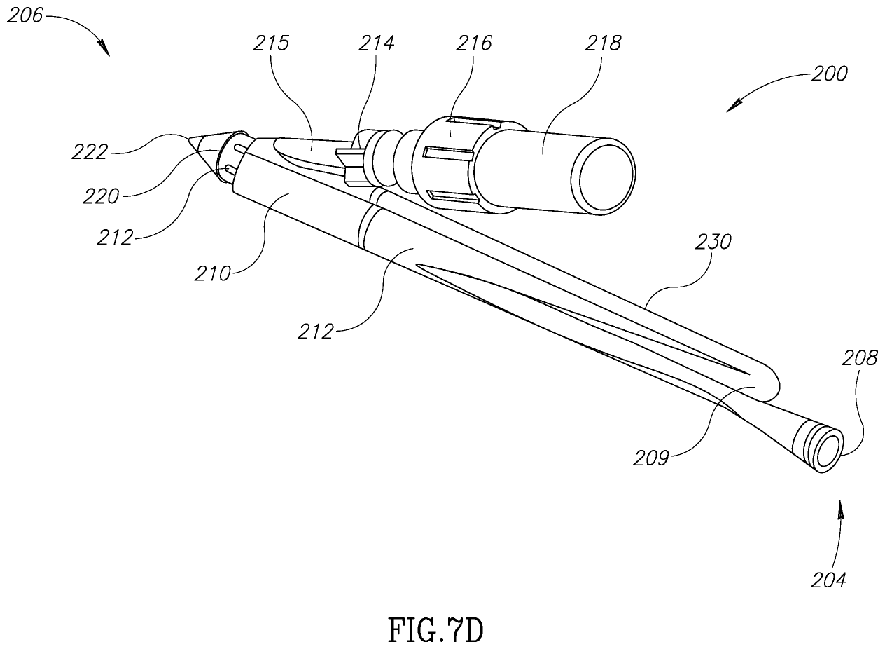

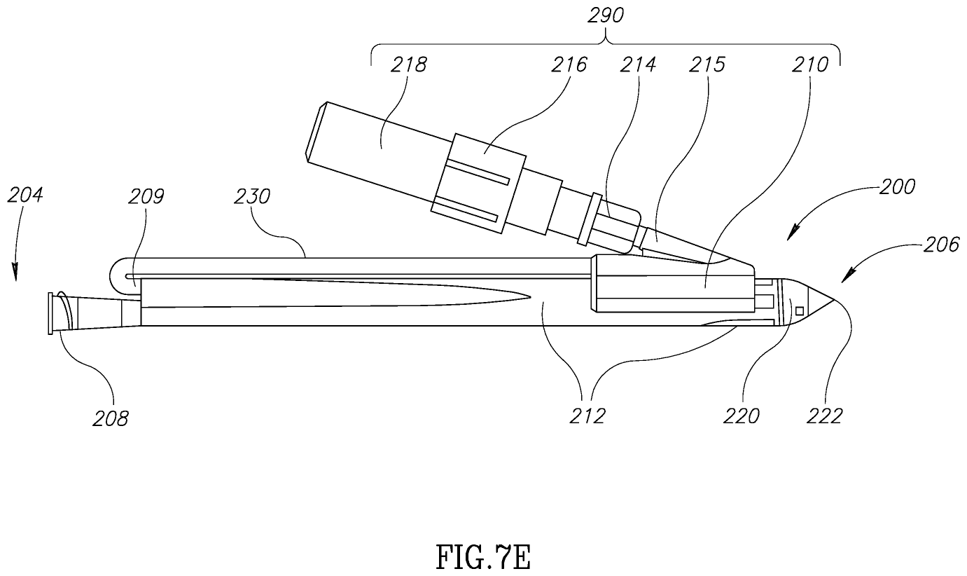

The housing of the device is in fluid flow communication with one or more containers or other vessels containing one or more therapeutic substances. The therapeutic substances may be provided in bottles, vials, ampoules, or any other suitable containers. The vessels/containers are removably affixed to and positioned on the housing via a therapeutic substance supply assembly as shown in FIGS. 7A-7E. The containers containing the therapeutic substances are generally single-use containers which contain predefined quantities and/or concentrations of therapeutic substances.

When the therapeutic liquid administered by the present invention is saline solution, the invention can be employed to clean a tissue surface. Subsequently, additional therapeutic substances, such as medications, nutrients, moisturizers or colorants may be administered. These therapeutic substances may be in liquid, emulsion or soluble powder form. Therapeutic substances such as platelet-rich plasma (PRP) mixtures may also be used as can other materials containing solids in a liquid carrier. This allows for more efficient dosing of the therapeutic substances, since, as will be appreciated by persons skilled in the art, the substances removed by cleaning, if left in place, would likely impede application and/or absorption of the desired therapeutic substances to the tissue undergoing therapeutic treatment.

The therapeutic substance supply assembly attached to the substantially tubular shaped housing of the device of the present invention may include control valves operative for introducing into the device a mixed flow of saline solution and other therapeutic substances. The valves can be used to obtain a desired concentration therein which can further be controlled, typically but without limiting the invention, by the operator during operation, to produce the mixed flow at specified times and for specified intervals. The device of the present invention would then accordingly produce a mixed therapeutic stream as desired and needed. Thus, as described above, a tissue surface could first be cleaned by saline solution and then dosed therapeutically with a medication solution when it is ready to optimally receive the dosage.

In an alternative embodiment of the device, instead of one mixed flow as mentioned hereinabove, the device of the present invention may be controlled and used to produce a number of therapeutic liquid flows for discharge into the elevated velocity gas flow. The therapeutic substances may also be turned on and off at specified times and for specified intervals. This arrangement also produces a mixed therapeutic stream as desired and needed. For example, the present invention can be used to treat a human scalp even where hair is present. First, the device provides an accelerated saline stream to clean the scalp of extraneous material, excess oils, and dead sloughed off epidermal tissue such as is known to produce dandruff. Then a moisturizing, nutrient, anti-dandruff, or anti-hair loss therapeutic substance is included in the accelerated stream to apply the desired therapeutic treatment to the scalp.

It should further be noted that the device is capable of applying the therapeutic substance to the desired tissue both topically and subcutaneously. Investigations employing prototype versions of the device have shown that the accelerated therapeutic stream produced will, for suitable droplet flow velocities and time of exposure of the tissue to the droplet flow, penetrate the tissue surface. This capacity of non-invasive subcutaneous treatment and dosage is a further advantage of the device.

It is contemplated that the device can also be used in lavage of hollow organs of the body.

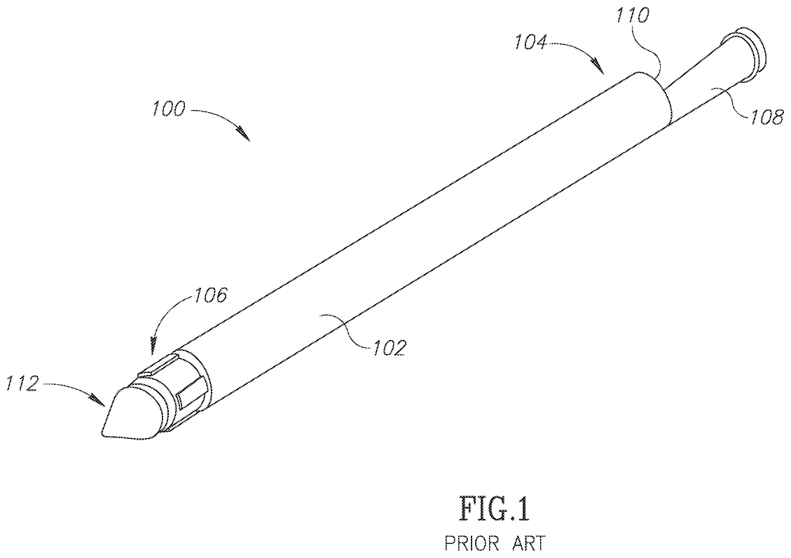

The discussion in conjunction with FIGS. 1-6 which follows is directed to an exemplary prior art stream jet delivery nozzle arrangement for accelerating a liquid/gas stream in the device of the present invention. In addition to the stream jet delivery nozzle arrangement shown in FIGS. 1-6, other jet delivery nozzle arrangements known in the art may also be used. The housing and control elements described and shown in FIGS. 1-6 are not necessarily the housing and control elements envisioned for use with the devices of the present invention. The housings and control elements of the devices of the present invention may be those described in conjunction with and shown in FIGS. 7A-7E. These may more typically be the housing and controls used.

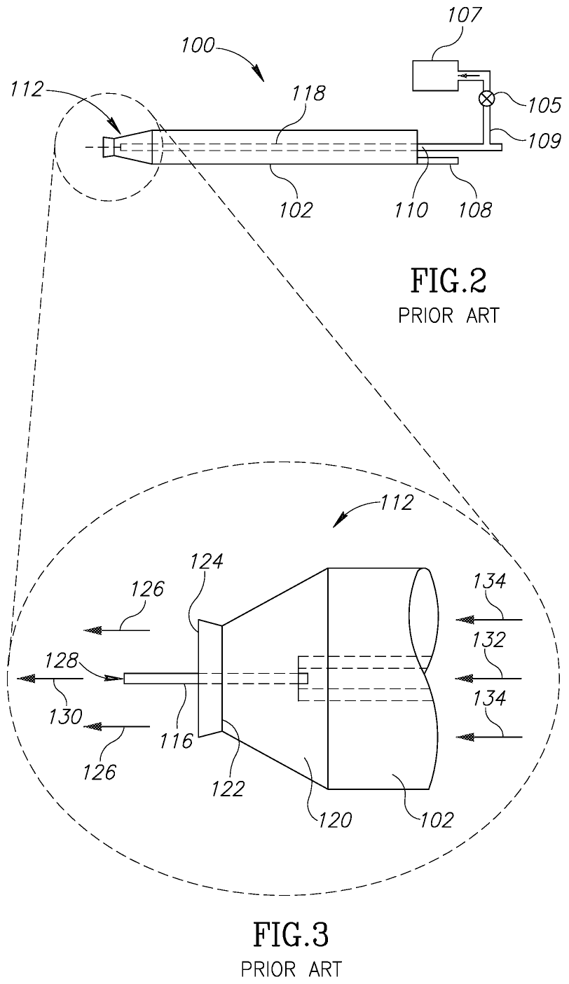

With reference to FIGS. 1 and 2, there is seen a device, referenced generally 100, for applying a high velocity liquid-gas therapeutic stream to tissue for therapeutic treatment thereof. Alternatively, the velocity of the stream may be regulated so as to merely provide cleansing of the tissue. Device 100 includes a housing portion referenced 102 having a generally tubular configuration, and having proximal and distal ends, referenced generally 104 and 106, respectively. A gas inlet port, referenced 108, and a liquid inlet port, referenced 110, are provided at proximal end 104, and a stream jet delivery nozzle arrangement referenced generally 112, is provided at distal end 106.

In FIG. 2, there is additionally shown, in schematic form, a therapeutic liquid inlet port 109 connecting pressurized therapeutic liquid source 107 via flow control element 105 to liquid inlet port 110 to allow production of a mixed flow of therapeutic liquid. It should be noted that the present arrangement producing one mixed therapeutic liquid flow is only shown by way of example, and that multiple therapeutic liquid flows, as well as control of the time of application of different therapeutic liquid flows are also contemplated as being part of the discussion herein.

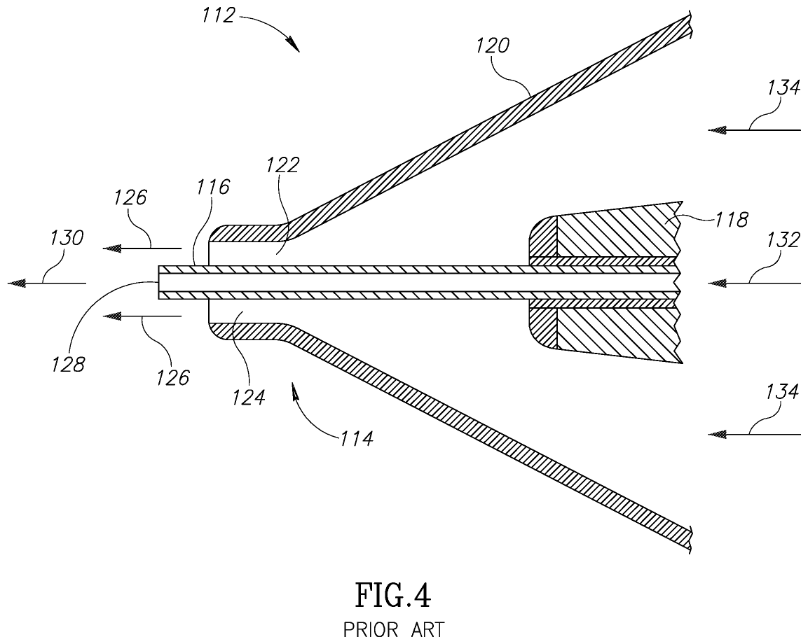

Referring now to FIGS. 3 and 4 in conjunction with FIG. 2, there are seen schematic and graphical cross-sectional views of nozzle arrangement 112 of device 100. Nozzle arrangement 112 includes a gas discharge nozzle referenced generally 114 and, disposed generally concentrically there-within, is a liquid discharge nozzle referenced 116. Liquid inlet port 110 (FIG. 2) is connected in fluid flow communication with liquid discharge nozzle 116 by means of a liquid communication tube referenced 118, disposed generally concentrically within tubular housing portion 102 (FIGS. 2 and 3).

Pressurized gas supplied from a pressurized gas source (not shown) enters device 100 through gas inlet port 108 (FIG. 2) and passes along and within tubular housing portion 102 as indicated by arrows 134, so as to discharge through gas discharge nozzle 114. Gas discharge nozzle 114 is generally configured having, in flow succession, a converging portion referenced 120, a throat portion referenced 122 and a diverging discharge portion referenced 124. The pressurized gas discharging from nozzle 114, as indicated by arrows 126, undergoes a rapid and substantial reduction in pressure to atmospheric pressure and a substantial acceleration to a high velocity, within the range of subsonic to supersonic velocity. Gas discharge nozzle 114 is configured such that the discharging gas has an average cone angle of less than 10 degrees, thereby providing a substantially parallel gas flow. The pressurized gas may be any benign gas such as nitrogen or even a mixture of gases such as s air.

Liquid, including therapeutic substances, from one or more pressurized therapeutic liquid sources (not shown) enters device 100 through liquid inlet port 110 (FIG. 2) and passes, as indicated by arrow 132, through liquid communication tube 118 (FIGS. 2 and 4). In turn, at distal end 106, therapeutic liquid is discharged through an opening referenced 128 in the distal end of liquid discharge nozzle 116 into the discharging flow 126 of gas, the therapeutic liquid flow being indicated by arrow 130.

It will be appreciated by persons skilled in the art that, as the pressurized discharging gas emerges 126 from gas discharge nozzle 114 into the atmosphere, it undergoes a rapid drop in pressure to atmospheric pressure. The sudden pressure drop results in a substantial acceleration of the velocity of the discharging gas flow that approximates or even exceeds the velocity of sound and results in the production of a shock wave The effect of the shock wave is to atomize the therapeutic liquid discharging from liquid discharge nozzle 116 into the flow of gas as a stream of therapeutic liquid droplets 130, such that there is obtained a relatively narrow jet of therapeutic liquid droplets in a high velocity gas flow 126.

Further, by way of example, the proportion of liquid flow to gas flow is extremely low due to the relatively high gas pressure of about 100 psi and low liquid pressure of about 2 psi, as well as the relatively large internal diameter of gas discharge nozzle 114 (about 0.5 mm) compared to a small internal diameter (about 0.09 mm) of liquid discharge nozzle 116. Consequently, little liquid tends to accumulate at the site to be cleaned or treated with one or more therapeutic substances. Furthermore, the relatively high gas flow has the effect of dispersing any accumulated liquid. When using a jet utilizing only liquid for cleansing, the liquid tends to accumulate on the tissue surface resulting in formation of a virtually stagnant liquid boundary layer close to and in contact with the surface, thereby reducing the effectiveness of cleansing. The very thin to negligible layer of liquid produced on the tissue surface by the above described nozzle arrangement allows more efficient dosage of additional therapeutic substances to the tissue surface, including the possibility of subcutaneous application of the therapeutic substances.

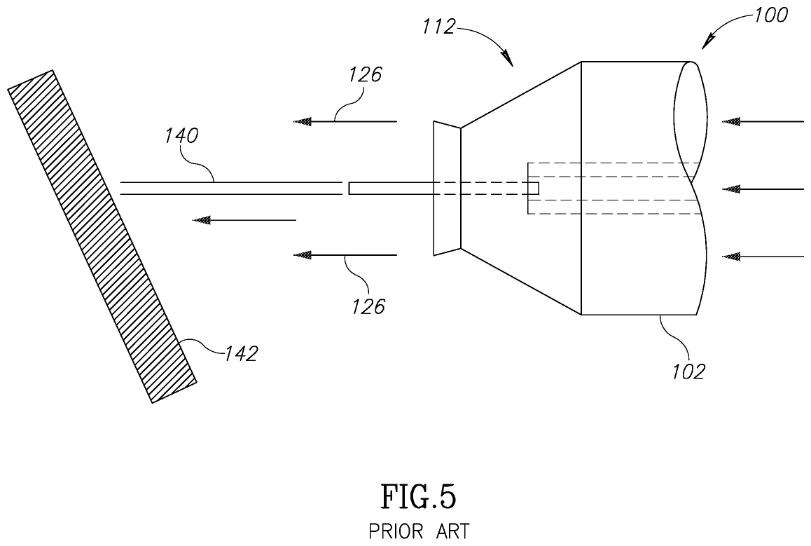

Referring now to FIG. 5, there is seen a high velocity flow of therapeutic liquid droplets referenced 140 discharging, in a high velocity gas flow 126, from nozzle arrangement 112 against a tissue surface referenced 142 to be cleaned and/or treated with therapeutic substances. Device 100 is held in the hand of a user by housing portion 102.

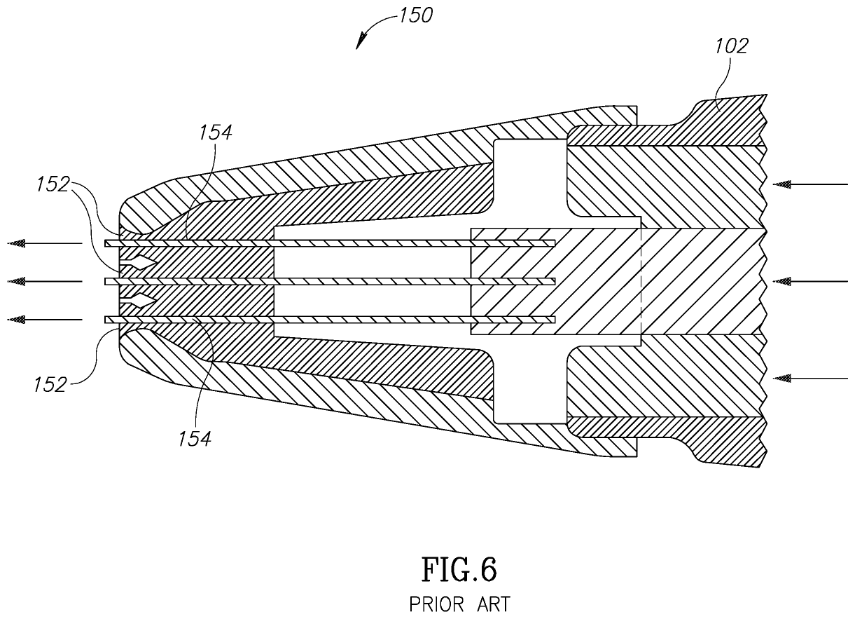

Referring now to FIG. 6, there is seen, according to an alternative construction of the above described device, a cross-sectional view of a device (not shown) having a housing portion 102 and a multiple nozzle arrangement referenced generally 150. Nozzle arrangement 150 is configured having multiple gas discharge nozzles referenced 152 and multiple therapeutic liquid discharge nozzles referenced 154 disposed generally concentrically within each gas nozzle 152 and projecting there-beyond. Such a multiple nozzle arrangement 150 facilitates increasing the rate of tissue cleaning, in the event that the system is used for this purpose. Additionally, as discussed below, the present configuration supports multiple therapeutic liquid flows, which may be individually controlled.

Referring now to FIGS. 7A-7C, there is seen, according to an embodiment of the present invention, a perspective, a side and a top view, respectively, of a device 200 constructed to provide one or more (in the Figures one or two) therapeutic substances in predefined dosages and/or concentrations to a patient being treated using the present invention. Without intending to limit the invention, therapeutic substances which may be used include saline solutions, medicaments, nutrients, moisturizers or mixtures of any of these. The housing and control elements in FIGS. 7A-7C (as well as those in FIGS. 7D-7E discussed below where only one therapeutic material is delivered) are different from the housing and control elements shown in FIGS. 1 and 2.

Nozzle arrangement 220, discharge nozzles 222 and hand piece housing portion 212 are constructed and configured substantially as described herein above and shown in FIGS. 1-6. Accordingly, description of these elements, their construction and their operation will not necessarily be repeated with respect to the embodiments of the invention presented and discussed in conjunction with FIGS. 7A-7E.

Two containers 218, such as, but without intending to limit the invention, bottles, vials or ampoules containing predefined dosages and/or concentrations of therapeutic liquid substances that are required in treating a patient, are positioned in container connectors 216. These containers 218 may be single-use containers. Container connectors 216 may be removably attachable and they may be single-use connectors. Container connectors 216 may be connected by luer locks 214 to liquid conduits 215 that lead to assembly base 210.

In some embodiments, there may be valves, such as stopcock valves 224, positioned between container connectors 216 and luer locks 214. It should be appreciated by persons skilled in the art that valves other than stopcock valves may also be used.

While luer locks generally are indicated throughout the discussion herein, it should readily be understood that other suitable connection fittings known to persons skilled in the art may also be used. In the claims, this element will generally be noted as “connection fittings” or “connection fitting”. Such designation is intended to include inter alia luer locks.

Assembly base 210, luer locks 214, stopcock valves 224, containers 218, container connectors 216, and liquid conduits 215 are typically, but with intending to limit the invention, made of rigid plastic. Housing portion 212 may also be formed of a rigid plastic. The exact plastics to be used for these elements are readily selectable by persons skilled in the art.

A side of assembly base 210 is disposed adjacent to device housing portion 212 and is shaped to conform to the adjacent side of housing portion 212. Assembly base 210 may be ultravioletly or ultrasonically bonded to housing portion 212. Alternatively, other methods of attachment known to persons skilled in the art suitable for use with plastics, such as adhesive gluing, may also be used.

Alternatively, in other embodiments, assembly base 210, luer lock 214, liquid conduit 215, stopcock valve 224 and container connector 216 may be constructed as an integral unit with handpiece housing portion 212 by using, for example, injection molding.

Container connectors 216, luer locks 214, liquid conduits 215, stopcock valves 224 and assembly base 210 collectively define, and will be herein referred to as a “therapeutic substance supply assembly” 290.

In some embodiments, such as the one discussed in conjunction with FIGS. 7D-7E below, there may be no need for stopcock valves. In such cases, the term “therapeutic substance supply assembly” 290 will be defined as previously but without the inclusion of stopcock or other valves.

More generally, a therapeutic substance supply assembly 290 is a structure attachable to a housing portion, such as element 212, including a container connector, such as element 216, for receiving a container, such as container 218. The structure allows container 218 to be in fluid flow communication with liquid discharge nozzles, such as discharge nozzles 222, of a nozzle arrangement, such as arrangement 220.

It should be understood that the specific construction of the therapeutic substance supply assemblies 290 shown in FIGS. 7A-7C and FIGS. 7D-7E are exemplary only. Other constructions may be used if they perform the functions of the assembly 290 as discussed herein.

Assembly base 210 is constructed and configured to fulfill two functions. First, it is configured to allow mounting of the therapeutic substance supply assembly 290 on housing portion 212. Second, assembly base 210 is formed with a conduit (obscured and not shown), herein often denoted as an “assembly base conduit”, allowing fluid flow communication between therapeutic substance supply assembly 290 and liquid inlet port 209 (discussed below).

The therapeutic substances in containers 218 are conveyed through container connectors 216 either under gravity or as a result of the therapeutic substances in container 218 being provided under pressure. A puncturing element (not shown) may be present in container connector 216. The puncturing element can puncture a cap of container 218 allowing the therapeutic substance to flow out of container 218 and ultimately into hand piece housing portion 212, as described below.

Stopcock valves 224 may be operated by the user to control flow of the therapeutic substance from containers 218 into housing portion 212. The operator may, by opening or closing stopcock valves 224, allow the therapeutic materials in one or both of therapeutic substance containers 218 to enter housing portion 212 and exit from nozzle arrangement 220 through liquid discharge nozzle(s) 222 (similar to elements 116 and 154 in, for example, FIGS. 4 and 7, respectively) at distal end 206 of device 200. The therapeutic liquid solution is then accelerated by pressurized gas exiting from gas discharge nozzles (similar to elements 114 and 152 in, for example, FIGS. 4 and 6, respectively) as discussed previously in conjunction with FIGS. 1-6.

The liquid therapeutic materials from containers 218 enter housing portion 212 of device 200 through liquid inlet port 209, the latter discussed in the paragraph immediately below. Liquid conduits 215 and the conduit formed in assembly base 210 (i.e. assembly base conduit-not shown) are in fluid flow communication with liquid inlet port 209. The liquid materials flow from the conduit formed in assembly base 210 (i.e. the assembly base conduit) through a flexible plastic tube 230 to port 209. From there, the liquid is transported either via flexible plastic tube 230 or liquid communication tube 118 (FIGS. 2 and 3) through housing portion 212 to discharge nozzle(s) 222 of nozzle arrangement 220.

It should readily be understood by persons skilled in the art that the flow of a therapeutic substance from a container 218 positioned in a container connector 216 of a therapeutic substance supply assembly 290 to nozzle arrangement 220 can occur using any suitable fluid flow communication arrangement.

FIGS. 7D and 7E show a device 200 similar to device 200 in FIGS. 7A-7C but having only a single therapeutic substance supply assembly 290. Elements in 7D-7E are similar to ones in FIGS. 7A-7C and have been numbered similarly. All elements in FIGS. 7D-7E are constructed and operated as discussed in conjunction with FIGS. 7A-7C and therefore will not be described again. In FIGS. 7D-7E, no stopcock valve is present. In other embodiments of FIGS. 7D-7E, valves, such as, but not limited to, stopcock valves, may be added.

It should readily be evident to one skilled in the art that devices, such as device 200, may also be configured to operate with more than two therapeutic substance container connectors 216 and/or more than two therapeutic substance supply assemblies.

Devices 200 may be used to apply the therapeutic droplet stream either topically or subcutaneously. Devices 200 may also be constructed to have a multiple nozzle configuration, similar to, for example, the one shown in and discussed hereinabove in conjunction with FIG. 6.

Most, if not all, of the device may be made of plastics having properties readily known to those skilled in the art.

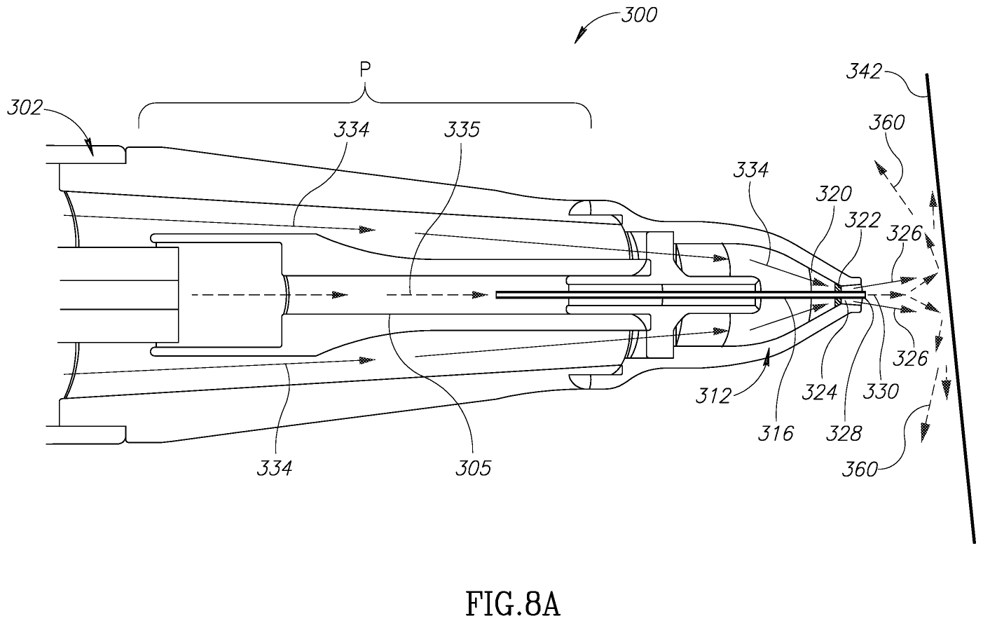

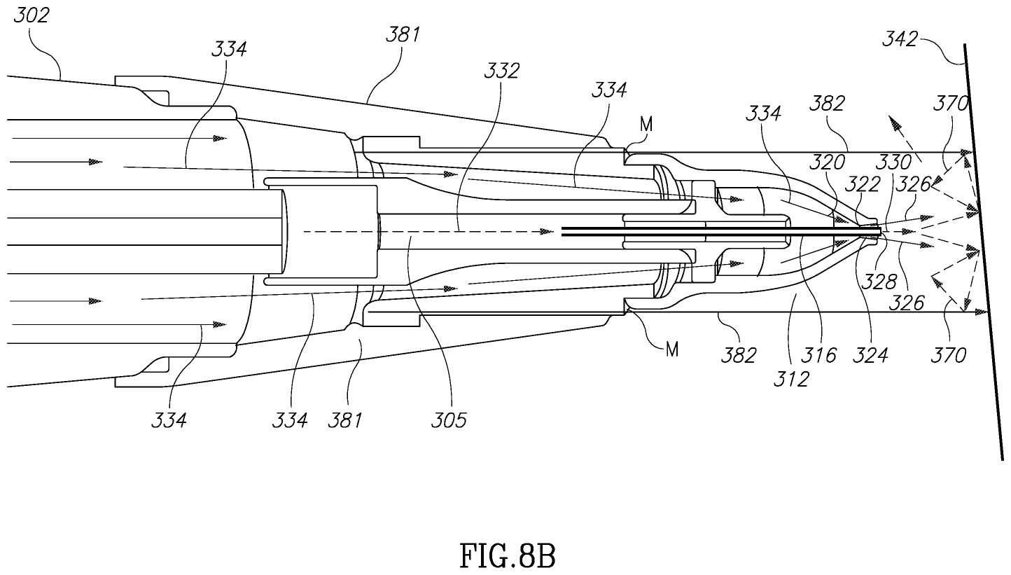

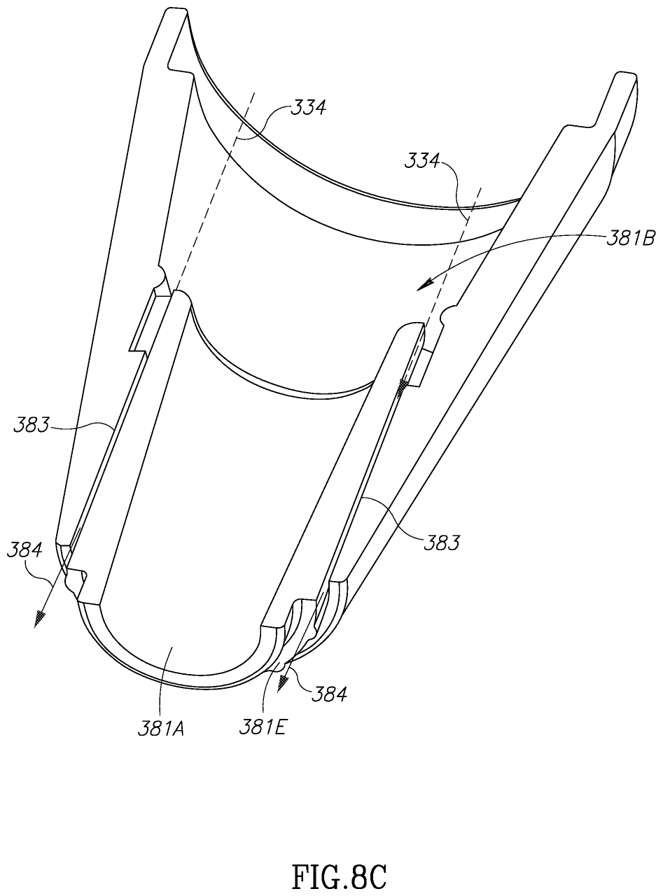

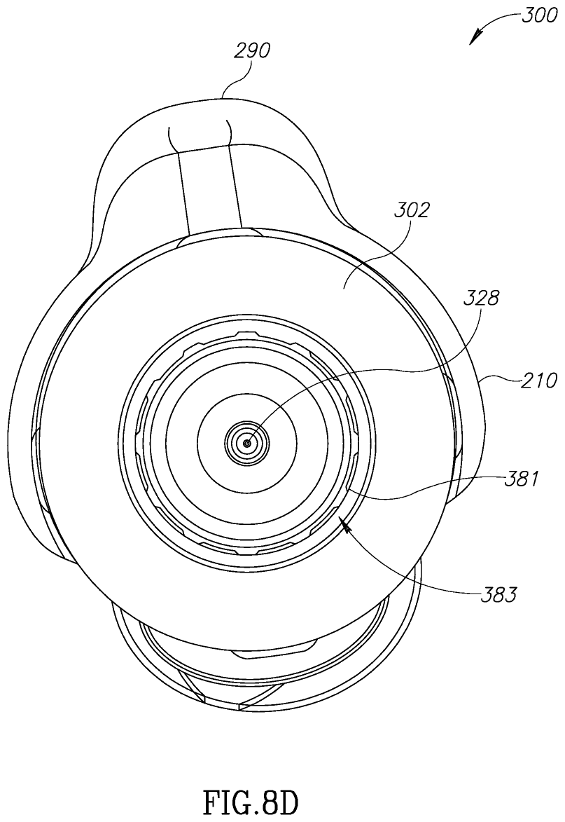

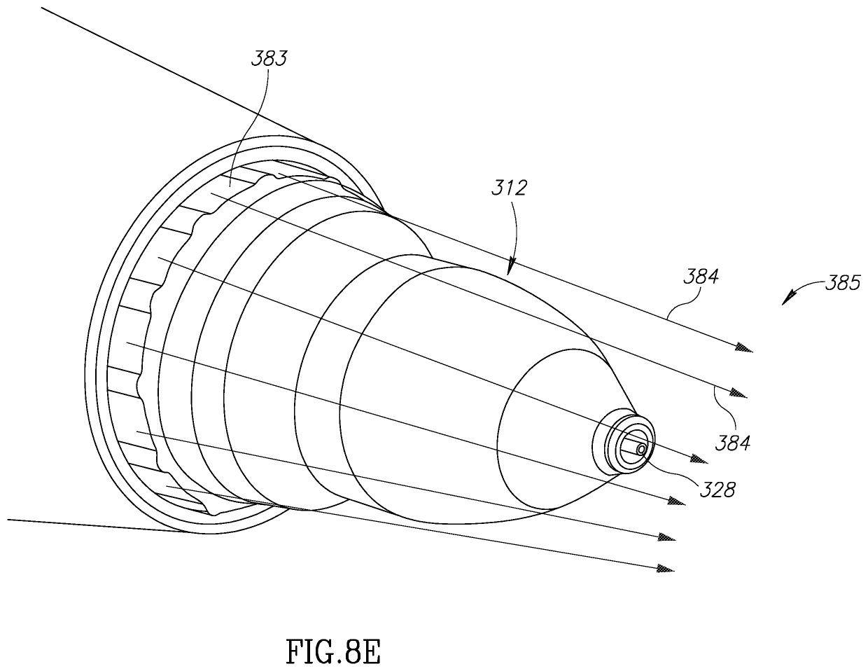

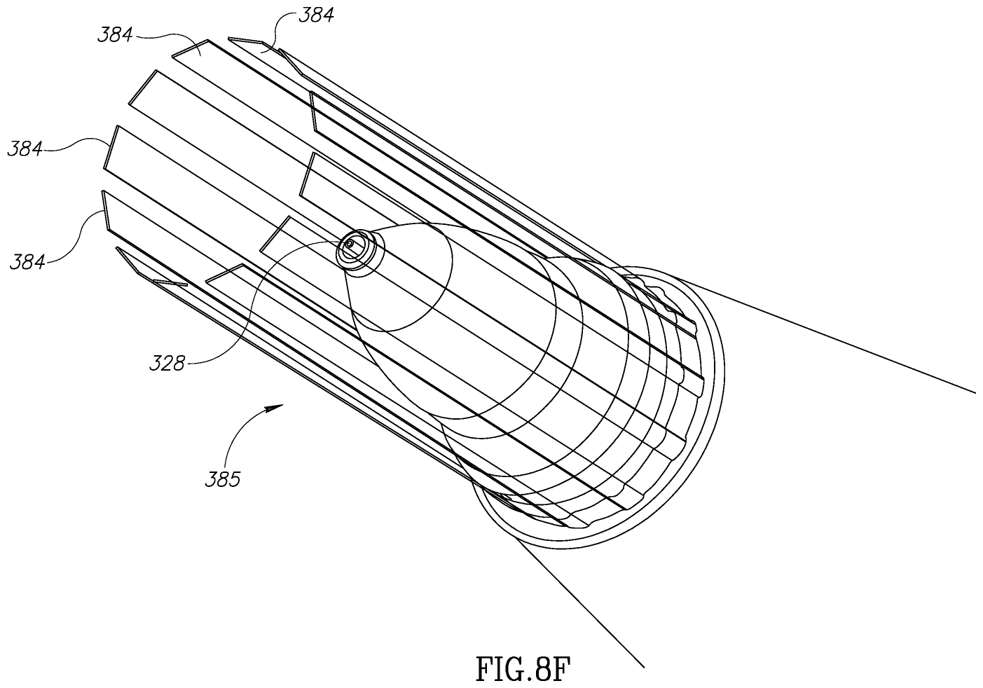

As shown in FIG. 8A, when the liquid emitted from the liquid nozzle 330 of device 300 is accelerated by the emitted gas stream 326, it disperses to a degree. As the microdroplets impact the target tissue, rebound microdroplets 370 rebound off the tissue treatment surface 342 further scattering the droplets. These rebounding microdroplets may often contain undesirable material such as blood which the user of the instrument wishes to avoid. This is true, for example, when platelet-rich plasma (PRP) is being administered subcutaneously by use of device 300. For the reason above, the present disclosure teaches a device having a shield (see FIGS. 8B and 8C) for preventing rebounding and scattering of the accelerated microdroplets in the direction of the user.

While solid barriers could serve as shields, solid barriers often interfere with the user's visibility of the tissue he is treating. Even relatively translucent materials, such as certain plastics and silicones known to persons skilled in the art, interfere with viewing the target tissue area being treated.

To overcome this problem, device 300 is equipped with a non-solid, non-continuous shield. Device 300 is constructed to provide a gas stream envelope 382 formed of shield gas streams 384 which acts as a gas stream shield 385 shown in FIGS. 8B-8F and discussed herein below. The numbering in FIGS. 8B-8F is the same as in FIGS. 1-5 with the first digit in the former changed to 3 while the first digit in the latter being 1. All elements in FIGS. 8A-8F constructed and operated as discussed in conjunction with FIGS. 1-5 will not be described again. Elements absent in FIGS. 1-5 but present in FIGS. 8A-8F have been given unused numbers in the range from 301 to 399.

A table of the elements in FIGS. 8A-8F and FIGS. 1-5 is presented below.

LEGEND FOR ELEMENTS

| FIGS. 8A | | FIGS. 1 |
|---|---|---|
| TO 8F | ELEMENT DESCRIPTION | to 5 |
| 302 | Device housing | 102 |
| 305 | Liquid delivery channel | |
| 312 | Nozzle arrangement | 112 |
| 314 | Gas discharge nozzle | |
| 316 | Liquid discharge nozzle/liquid emitting microtube | 116 |
| 320 | Converging portion of nozzle arrangement | 120 |
| 322 | Throat portion of nozzle arrangement | 122 |
| 324 | Diverging portion of nozzle arrangement | 124 |
| 326 | Discharge gas flow | 126 |
| 328 | Opening/tip of liquid discharge nozzle(s) | 128 |
| | (microtube) | |
| 330 | Discharged (therapeutic) liquid microdroplets | 130 |
| 332 | Incoming liquid from liquid supply source | 132 |
| 334 | Incoming gas from gas supply source | 134 |
| 342 | Treatment surface | 142 |
| 360 | Scattering (dispersing) microdroplets | |
| 370 | Rebounding microdroplets | |
| 381 | Gas stream shield generator | |
| 381A | Gas shield generator- insert portion | |
| 381B | Gas shield generator-wall portion | |
| 381E | Edge of gas stream shield generator | |
| 382 | Gas stream shield envelope | |
| 383 | Gas stream openings/holes/slits | |
| 384 | Shield gas streams | |
| 385 | Gas stream shield | |

Reference is now made to FIG. 8A where a cut-away side view of device 300 is shown. As noted, elements in FIG. 8A which are constructed and operated as in FIGS. 1-6 will not be described again. FIG. 8A is essentially equivalent to the devices in FIGS. 1-6. In the latter, the therapeutic substance supply assembly is not shown.

FIG. 8A shows and emphasizes the dispersal and scattering of the accelerated microdroplets 360 after being emitted from liquid discharge nozzle 316. This scattering, if not controlled, allows some microdroplets to travel in the direction toward the proximal end of device 300, possibly even reaching the user. As already noted, this is undesirable particularly when hazardous materials such as blood are used.

Turning to FIG. 8B a cut-away side view of device 300 is again presented. The only element present in FIG. 8B absent in FIG. 8A is a gas stream shield generator 381, constructed to curb scattering and dispersion resulting from the rebounding of microdroplets in the direction of the user.

Shield generator element 381 generates gas streams 384 which flow in the direction toward the tissue surface 342 being treated. When taken together, gas streams 384 form a gas stream envelope 382 (see FIGS. 8B, 8E and 8F). The gas stream envelope 382 is best seen in FIGS. 8B, 8E and 8F. The envelope serves to cage the microdroplets so that few, if any, reach the user. Shield gas streams 384 being directed in a distal direction away from device 300 force the microdroplets generated by nozzle arrangement 312 to move in a generally distal direction away from the user. Gas stream shield 385 reduces the scattering and dispersion of the microdroplets resulting from their impact with tissue surface 342.

Gas stream shield generator 381 includes a plurality of slits 383 with openings to the ambient at shield generator distal edge 381E (FIG. 8C). Gas 334 arriving from an external gas supply (the source not shown) after passing through slits 383 and exiting the slits at 381E form a plurality of gas streams 384. When taken together, gas streams 384 exiting shield generator 381 at relatively high pressure at points M of generator 381 form a gas stream envelope 382 shown in FIGS. 8B, 8E and 8F. Envelope 382 effectively traps and “cages” the microdroplets within the cylinder formed by gas streams 384 as seen in FIG. 8B and forces them to move in a distal directions preventing scattering.

Turning now to FIG. 8C, a partially cut-away view of gas stream shield generator 381 is shown. Gas stream shield generator 381 is disposed around the hand piece 302 or proximal portion of the nozzle in the region indicated by P shown in FIG. 8A.

In FIG. 8C, gas stream shield generator 381 is shown to consist initially of two parts. One part 381A of generator 381 is an insert placed within a second part 381B; the two parts are then glued or welded together using ultraviolet or ultrasonic welding. The entire generator 381 is attached to the hand piece at region P (shown in FIG. 8A) with part 381B forming part of the hand piece 302 wall or part of the nozzle arrangement 312 wall.

Insert 381A is positioned within wall section 381B and constructed so that there are a plurality of identical spacers circumscribing the exterior side of the insert. The exterior side of the insert here refers to the side closest to part 381B. These spacers space insert 381A from wall section 381B, thereby generating the plurality of slits through which the pressurized inlet gas passes. Elements 381A and 381B are typically formed of suitable plastics known to persons skilled in the art.

In other embodiments, gas shield generator 381 may be formed as a single integral element made by injection molding.

The gas for shield generator 381 may be supplied by the same source as that which supplies the gas passing through nozzle arrangement 312 (FIG. 8A).

From FIG. 8C it can readily be understood that gas 334 is delivered from a gas supply source (not shown) and enters slits 383. The gas exits the slits at the openings at of the distal end 381E of gas stream shield generator 381. At that point, the flowing gas is denoted as shield gas streams 384.

In some embodiments, the high pressure gas source (not shown) is the same source which supplies the nozzle arrangement of device 300 of FIGS. 8A and 8B. In such a case, the gas flow from the source is activated by a single valve or other actuator element.

In other embodiments, the source of gas for the gas stream shield may be a source different from that which forms the high velocity mist exiting from nozzle 316. In such an embodiment, there are two separate activators, each activating a gas flow from a different source.

Gas stream shield generator 381 is attached to device housing 302. There can be many different means of attachment of shield 385 to device housing 302 or proximal region of nozzle arrangement 312. Without intending to limit the invention, these may include ultraviolet bonding using polymeric materials.

The number of slits 383 through which gas is emitted forming gas stream shield 385 can be any plurality of slits, for example, 2 to 16 slits, preferably 12, as in the present Figures. The thickness of the slits can be in a range between 0.05 millimeters (“mm”) and 0.3 mm, preferably 0.1 mm. Slits 383 may have a surface area within a range between 0.075 millimeters squared (“mm²”) and 0.5 mm², preferably 0.14 mm². The shape of the slits in the attached Figures have circular arc section shapes but hexagonal and other such shapes may also be used.

The emitted gas streams 384 form a discontinuous envelope 382 (the discontinuity of the envelope and shield can best be seen in FIGS. 8E and 8F) substantially in the shape of a right circular cylinder surrounding the dispersing microdroplets.

While FIGS. 8B-8F show a device 300 employing a single nozzle (micro-tube) 316 delivering the liquid and/or therapeutic solutions, in other embodiments a plurality of such nozzles (micro-tubes) may be used, similar to the embodiment in FIG. 6.

The shield of the device in FIGS. 8B-8F is effective independent of the angle made by the longitudinal axis of device 300 and the tissue being treated i.e. the angle of attack of the droplet stream. Angle of attack as used here can be thought of as the angle between a body's reference line and the oncoming fluid flow.

It can readily be understood that when the angle of attack is not 90° there is a deviation from the right circular cylinder discussed above. This deviation does not materially affect the desired operation of gas stream shield 385.

While not clearly observable in all of FIGS. 8A-8F, it should readily be understood that, as in FIGS. 1-7E, liquid discharging nozzle 316 extends past the gas nozzles 314 in the devices shown in FIGS. 8A -8F.

In addition to preventing “splash” of droplets on the user, it is envisioned that another benefit of employing gas stream shield 385 would be a reduction in the amount of therapeutic substance used. This can be attributed to less wasted therapeutic substance because of the presence of the restraining gas stream shield.

It will be appreciated by persons skilled in the art that the present invention is not limited by the drawings and description hereinabove presented. Rather, the invention is defined solely by the claims that follow.

What is claimed is:

1. A device for administering a therapeutic substance to tissue for use with a pressurized gas source, and including:
a) a housing having a liquid therapeutic substance inlet port;
b) a gas inlet port connected to the pressurized gas source;
c) a stream jet delivery nozzle arrangement in fluid flow communication with said gas inlet port and in fluid flow communication with said therapeutic substance inlet port, the therapeutic substance being discharged from said stream jet delivery nozzle arrangement into an elevated velocity flow of gas discharged from said delivery nozzle arrangement where upon said substance forms microdroplets which when impinging upon the tissue to be treated rebounds and disperses therefrom; and
d) a gas stream shield generator including a plurality of slits, wherein pressurized gas passes through the slits providing gas streams external to the nozzle arrangement, the gas streams forming an envelope that contains the dispersal of the rebounded droplets resulting from impinging on the tissue thereby shielding the user.

2. A device according to claim 1, wherein said gas stream shield generator includes an insert and a wall section of the housing or a wall section of the proximal portion of the nozzle arrangement, the insert disposed within the wall section and constructed so that there are a plurality of identical spacers circumscribing an exterior side of the insert spacing the insert from the wall section, thereby generating the plurality of slits through which the pressurized gas passes.

3. A device according claim 1, wherein the number of slits are from 2 to 16 slits.

4. A device according to claim 2, wherein the plurality of slits are symmetrically disposed on the distal edge of the shield generator insert, each slit equidistant from its nearest neighbor slits.

5. A device according to claim 1, wherein the area of each slit is between 0.075 mm² and 0.5 mm².

6. A device according to claim 5, wherein the area of each slit is between 0.1 mm² and 0.2 mm².

7. A device according to claim 1, wherein the slits are shaped as circular arc sections.

8. A device according to claim 1, further including at least one therapeutic substance supply assembly mounted onto said housing, each therapeutic substance supply assembly configured for receiving at least one container containing a predefined quantity or concentration of liquid therapeutic substance.

9. A device according to claim 8, wherein said liquid therapeutic substance inlet port is in fluid flow communication with said therapeutic substance supply assembly and also in fluid flow communication with said stream jet delivery nozzle arrangement.

10. A device according to claim 1, wherein said stream jet delivery nozzle arrangement includes:
i) at least one gas discharge nozzle arranged to receive a flow of pressurized gas from said gas inlet port and configured to accelerate the flow of gas so as to discharge it at an elevated velocity; and,
ii) at least one liquid discharge nozzle arranged to receive a flow of liquid therapeutic substance from a therapeutic substance supply assembly and operative to discharge the flow of therapeutic substance into the elevated velocity flow of gas,
thereby accelerating the velocity of the discharged liquid therapeutic substance as a stream of accelerated therapeutic droplets and to discharge the stream of accelerated therapeutic droplets towards a tissue mass for treatment by the therapeutic substance.

11. A system for administering a therapeutic substance to tissue, including:
a) a pressurized gas source;
b) at least one container containing a predefined quantity or concentration of a liquid therapeutic substance; and
c) a device which includes:
(i) a housing having a liquid therapeutic substance inlet port;
(ii) a gas inlet port connected to said pressurized gas source;
(iii) a stream jet delivery nozzle arrangement in fluid flow communication with said gas inlet port and in fluid flow communication with said liquid therapeutic substance, the liquid therapeutic substance being discharged from said stream jet delivery nozzle arrangement into an elevated velocity flow of gas discharged from said delivery nozzle arrangement which upon impinging the tissue to be treated rebounds and disperses therefrom; and
(iv) a gas stream shield generator including a plurality of slits, wherein pressurized gas passes through the slits providing gas streams external to the nozzle arrangement, the gas streams forming an envelope that reduces dispersal of the droplets resulting from impinging on the tissue, thereby shielding the user.

12. A gas stream shield generator including a wall of a nozzle arrangement and/or hand piece with an insert disposed therein for producing a plurality of slits therebetween, the generator having a generally truncated conical shape with a wider end proximal to a gas source and a narrower end distal from the gas source, the slits having openings to the ambient at the distal end of the generator where pressurized gas passes through the slits providing gas streams external to the nozzle arrangement, the gas streams forming an envelope that reduces dispersal of microdroplets generated by the nozzle arrangement after rebounding of the microdroplets from tissue being treated thereby shielding a user from the liquid.
